(19) United States
(12) Patent Application Publication
(10) Pub. No.: US 2021/0261511 A1
(43) Pub. Date: Aug. 26, 2021

(54) GENETICALLY MODIFIED BACTERIAL CELLS AND METHODS USEFUL FOR PRODUCING TETRAMETHYL PYRAZINE

(71) Applicant: The Regents of the University of California, Oakland, CA (US)

(72) Inventors: Aindrila Mukhopadhyay, Oakland, CA (US); Thomas T. Eng, Berkeley, CA (US)

(21) Appl. No.: 17/185,196

(22) Filed: Feb. 25, 2021

Related U.S. Application Data
(60) Provisional application No. 62/982,001, filed on Feb. 26, 2020.

(51) Int. Cl. C07D 241/04; C12N 1/20; C12N 15/52

(52) U.S. Cl. CPC C07D 241/04; C12N 2510/00; C12N 15/52; C12N 1/20

(57) ABSTRACT

The present invention provides for a genetically modified bacterial host cell comprising one or more enzymes of a heterologous isoprenoid or isopentenol production pathway and capable of producing tetramethyl pyrazine.

### CROSS REFERENCE TO RELATED APPLICATIONS

This application claims priority to U.S. Provisional Patent Application Ser. No. 62/982,001, filed Feb. 26, 2020, which is incorporated by reference in its entirety.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of the production of tetramethyl pyrazine.

### REFERENCE TO A “SEQUENCE LISTING” SUBMITTED AS ASCII TEXT FILES VIA EFS-WEB

The Sequence Listing written in file 2018-101-02_Sequence_Listing_ST25.txt created on Feb. 22, 2021, 13,437 bytes, machine format IBM-PC, MS-Windows operating system, in accordance with 37 C.F.R. §§ 1.821- to 1.825, is hereby incorporated by reference in its entirety for all purposes.

### BACKGROUND OF THE INVENTION

Alkylpyrazines are naturally distributed, heterocyclic aromatic compounds with a nutty or roasted flavor profile (Masuda and Mihara, 1988). Of the alkylpyrazine compounds, 2,3,5,6-tetra-methylpyrazine or tetramethyl pyrazine (also known as TMP or ligustrazine) is a biologically active alkaloid commonly found in the rhizobiome of Ligusticum wallichii (Ke-ji et al., 1983). It has been used as a flavoring agent (Zhu et al., 2010), studied as a potentially pharmacologically active molecule (Chen et al., 2018; Ferreira and Kaiser, 2012; Kao et al., 2013), or applied onto surfaces as a coating (Lee, 2014; Ng, 2017). While TMP has been examined for potential use in many fields, its widespread evaluation is hindered due to its limited commercial availability. Chemical synthesis routes to TMP involve multiple synthesis steps and are inefficient (Dickschat Jeroen et al., 2010; Deng et al., 2006). Commercially available TMP is often purified through an ethanol-ethyl ether extraction from plants such as Ephedra sinica (Li et al., 2001). More sustainable, biological routes towards TMP production have focused on optimizing cultivation conditions of Bacillus subtilis, which can naturally produce the TMP precursor, acetoin (Kosuge and Kamiya, 1962; Xiao et al., 2014). TMP is inefficiently generated from acetoin (26% yield) by heating the cell lysate to ˜65° C. for 2 hours (Xiao et al., 2014). Outside of B. subtilis, trace amounts of alkylpyrazines (such as TMP) have been detected in the headspace of Corynebacterium glutamicum (Dickschat Jeroen et al., 2010), but evidence of higher titers of TMP has only been reported from a single C. glutamicum isolate (MB-1923) which is not publicly available and has no associated genomic metadata (Demain et al., 1967).

U.S. Pat. Nos. 8,871,843 and 9,765,168 disclose the use of TMP as a surface coating related to flame retardant processing. Chinese Patent No. ZL200310102368.0 (Publication No. CN1238344C) discloses a chemical synthesis of TMP.

### SUMMARY OF THE INVENTION

The present invention provides for a genetically modified bacterial host cell capable of producing tetramethyl pyrazine comprising one or more enzymes of a heterologous isoprenoid or isopentenol production pathway. In some embodiments, the genetically modified bacterial host cell is also capable of producing isopentenol.

In some embodiments, the one or more enzymes of a heterologous isoprenoid or isopentenol pathway comprise the enzymes described herein, including but not limited to the enzymes encoded by the genes described in Table 1. In some embodiments, the one or more enzymes of a heterologous isoprenoid or isopentenol pathway comprise the enzymes: acetyl-CoA acetyltransferase (AtoB), hydroxymethylglutaryl-CoA synthase (HMGS), 3-hydroxy-3-methylglutaryl-CoA reductase (HMGR), mevalonate kinase (MK), phosphomevalonate decarboxylase (PMD), phosphatase, or any homolog thereof having the same enzymatic activity. In some embodiments, the enzymes are obtained from any suitable fungal (such as yeast, such as S. cerevisiae) or bacterial species. In some embodiments, the MK is Staphylococcus aureus MK or Corynebacterium kroppenstedtii MK. In some embodiments, the HMGR is a Staphylococcus aureus HMGR homolog or Corynebacterium kroppenstedtii HMGR homolog.

In some embodiments, the bacterial host cell is an actinobacteria cell. In some embodiments, the actinobacteria cell is an Actinomycetales cell. In some embodiments, the Actinomycetales cell is a Corynebacterineae cell. In some embodiments, the Corynebacterineae cell is a Corynebacteriaceae cell. In some embodiments, the Corynebacteriaceae cell is a Corynebacterium cell. In some embodiments, the Corynebacterium cell is Corynebacterium glutamicum, Corynebacterium kroppenstedtii, Corynebacterium alimapuense, Corynebacterium amycolatum, Corynebacterium diphtherias, Corynebacterium efficiens, Corynebacterium jeikeium, Corynebacterium macginleyi, Corynebacterium matruchotii, Corynebacterium minutissimum, Corynebacterium renale, Corynebacterium striatum, Corynebacterium ulcerans, Corynebacterium urealyticum, or Corynebacterium uropygiale. In some embodiments, the Corynebacterium glutamicum is strain ATCC 13032. In some embodiments, the bacterial host cell comprises native genes encoding the following enzymes: ALS, acetolactate synthase (ilvB, Cgl1271; ilvN, Cgl1272) and/or AR, acetoin reductase (butA, Cgl2674).

The present invention provides for a method for a genetically modified bacterial host cell producing tetramethyl pyrazine (TMP), comprising (a) providing a genetically modified bacterial host cell of the present invention, (b) culturing or growing the host cell in a suitable culture or medium such that TMP is produced and optionally isopentenol is produced, and (c) optionally extracting or separating the TMP and optionally isopentenol from the rest of the culture or medium, and/or host cell.

In some embodiments, the providing step (a) comprises introducing a nucleic acid encoding the one or more enzymes of a heterologous isoprenoid or isopentenol production pathway operatively linked to a promoter capable of expressing the one or more enzymes of a heterologous isoprenoid or isopentenol production pathway in the host cell into the host cell. In some embodiments, the culturing or growing step (b) comprises the host cell growing by respiratory cell growth. In some embodiments, the culturing or growing step (b) takes place in a batch process or a fed-batch process, such as a high-gravity fed-batch process. In some embodiments, the culture or medium comprises a rich medium, such as LB (Lysogeny-Broth) or comprising one or more ingredients of LB, such as tryptone and/or yeast extract. In some embodiments, the culture or medium comprises hydrolysates derived or obtained from a biomass, such as a lignocellulosic biomass. In some embodiments, the culture or medium comprises one or more carbon sources, such as a sugar, such as glucose or galactose, or glycerol, or a mixture thereof. In some embodiments, the carbon source is fermentable. In some embodiments, the carbon source is non-fermentable. In some embodiments, the culture or medium comprises urea as a nitrogen course. In some embodiments, the culture or medium comprises an ionic liquid (IL).

In some embodiments, the method results in the genetically modified bacterial host cell producing equal to or more than about 1 g/L, 1.5 g/L, 2 g/L, 2.5 g/L, 3 g/L, 3.5 g/L, 4 g/L, 4.5 g/L, or 5 g/L of TMP.

The present invention provides for a method for constructing a genetically modified bacterial host cell of the present invention, comprising (a) introducing a nucleic acid encoding the one or more enzymes of a heterologous isoprenoid or isopentenol production pathway operatively linked to a promoter capable of expressing the one or more enzymes of a heterologous isoprenoid or isopentenol production pathway in the host cell into the host cell.

In some embodiments, the one or more enzymes of a heterologous isoprenoid or isopentenol production pathway is heterologous to the host cell. In some embodiments, the one or more enzymes of a heterologous isoprenoid or isopentenol production pathway comprises one or more fungal or bacterial enzymes of a heterologous isoprenoid or isopentenol production pathway. In some embodiments, the one or more enzymes of a heterologous isoprenoid or isopentenol production pathway comprises an amino acid sequence that is at least 70%, 80%, 90%, 95%, or 99% identical to the amino acid sequence of SEQ ID NO:1-4.

In some embodiments, the invention comprises the use of a heterologous codon-optimized version of the one or more enzymes of a heterologous isoprenoid or isopentenol production pathway in a bacterial host cell.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Diagram of Proposed Tetra-methylpyrazine (TMP) Production Pathway in C. glutamicum Model. Pyruvate is generated from glucose using the Embden-Meyerhof-Parnas (EMP) pathway and converted to TMP in four steps as indicated. ALS, acetolactate synthase (ilvB, Cgl1271; ilvN, Cgl1272); AR, acetoin reductase (butA, Cgl2674). The upper-case letter S with arrows represents non-enzymatic spontaneous reactions (see Xiao et al, 2014).

FIG. 2. mk and hmgR Variants from bacterial species bias production towards tetra-methylpyrazine. Top panel. Schematic of the heterologous isopentenol production pathway. The two genes selected for optimization by substitution with homologs are indicated with an asterisk (*). Bottom panels. Analysis of the engineered isopentenol production pathway in C. glutamicum using mk and hmgR homologs from S. aureus and C. kroppenstedtii in a 24 well plate format. C. glutamicum ΔpoxB ΔldhA strain harboring the original isopentenol production pathway (JBEI-19559) was compared with variants where the S. cerevisiae MK and HMGR are replaced with mk and hmgR from S. aureus (JBEI-19652) or C. kroppenstedtii (JBEI-19658). Samples are cultivated in CGXII media 4% D-glucose in a 24 well plate format. TMP or isopentenol titers are analyzed at the timepoints indicated and are an average of three biological replicates. The error bars represent standard error.

FIG. 3A. Proteomic Analysis of Engineered C. glutamicum strains. Hierarchical clustering of proteins enriched after heterologous gene pathway expression in engineered C. glutamicum strains.

FIG. 3B. Proteomic Analysis of Engineered C. glutamicum strains. Gene functions of enriched proteins as modeled with eggNOG-mapper to assign genes into categories of orthologous groups (COGs). WT C. glutamicum (JBEI-7936) is compared against strains harboring the original isopentenol pathway (JBEI-19571) or the plasmid variants (JBEI-19652 and JBEI-19658). This figure shows the broad categories of gene function.

FIG. 3C. Proteomic Analysis of Engineered C. glutamicum strains. Gene functions of enriched proteins as modeled with eggNOG-mapper to assign genes into categories of orthologous groups (COGs). WT C. glutamicum (JBEI-7936) is compared against strains harboring the original isopentenol pathway (JBEI-19571) or the plasmid variants (JBEI-19652 and JBEI-19658). This figure shows the distribution of proteins into specific COGs. COGs falling into related categories from the top panel are grouped together in brackets below. COG definitions are defined in Galperin et al, 2014.

FIG. 4A. GC/MS analysis of tetra-methylpyrazine and S-acetoin. Identification of tetra-methylpyrazine and other peaks by GC-MS analysis. The genotypes of the strains used are the same as described in FIG. 1. Peak identification. 1. Acetoin (3-hydroxy-2-butanone). 2. Isopentenol (3-methyl-3-buten-1-ol). 3. 4-penten-1-ylacetate. 4. 2,3,5-tri-methyl-pyrazine. 5. 2,3,5,6-tetra-methyl-pyrazine. 6. 3,5-diethyl-2-methyl-pyrazine.

FIG. 4B. GC/MS analysis of tetra-methylpyrazine and S-acetoin. Comparison of acetoin peak height across different engineered strains by GC/MS analysis.

FIG. 4C. GC/MS analysis of tetra-methylpyrazine and S-acetoin. Assessment of spontaneous conversion of acetoin or diacetyl to TMP in the absence of cells using 100 mM of either starting compound spiked into CGXII growth media. No TMP is detected.

FIG. 5. Specific forms of ionic liquids induce tetra-methylpyrazine production in C. glutamicum. The production profiles of the C. glutamicum (JBEI-19572) against three types (imidizolium, cholinium, and protic form) of ionic liquids are examined in CGXII media with 4% D-glucose in a 24 well plate format. ([C₂C₁im]⁺); cholinium ([Ch]⁺); ethanolamine acetate [ETA][OAc]; and diethanolamine acetate [DEOA][OAc] A. Produced titers of tetra-methylpyrazine are shown at 48 hours post induction under 75 mM (A) or 150 mM (B) [C₂C₁im][OAc], [Ch][OAc], [ETA][OAc], and [DEOA][OAc]. The control experiment is performed without IL supplementation (No ILs). Data shown are an average of biological triplicates, and the error bars represent standard error.

FIG. 6. Fed-batch mode production of TMP in engineered C. glutamicum. C. glutamicum (JBEI-19572) is initially cultivated in batch mode using CGXII minimal media including 8% starting D-glucose and 50 mM [Ch][Lys] in a 2 L Sartorius Bioreactor. Subsequently, fed-batch mode is initiated when the initial D-glucose concentration decreased below 1%. A pulse mode feeding strategy is utilized to raise the D-glucose concentration above 1%. Final products and organic acid concentrations are quantified and are indicated as labeled in the legend above.

FIG. 7A. Extraction efficiency of TMP. Extraction efficiency of TMP using ethyl acetate as the extraction solvent. The linear range of TMP dissolved in ethyl acetate is plotted on the right.

FIG. 7B. Extraction efficiency of TMP. Extraction efficiency of TMP using dichloromethane. The linear range of TMP dissolved in dichloromethane is plotted on the right.

FIG. 7C. Extraction efficiency of TMP. Impact of exogenous ionic liquid on wild-type C. glutamicum. Without the presence of a heterologous gene pathway, no TMP was detected with any ionic liquid treatment.

FIG. 8. Mass fragmentation pattern of acetoin and TMP.

FIG. 9. Quantification of isopentenol production in production strains treated with exogenous ionic liquids. The data shown are an average of biological triplicates, and the error bars represent standard error.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

The term “about” refers to a value including 10% more than the stated value and 10% less than the stated value.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The terms “host cell” is used herein to refer to a living biological cell that can be transformed via insertion of an expression vector.

The term “heterologous” as used herein refers to a material, or nucleotide or amino acid sequence, that is found in or is linked to another material, or nucleotide or amino acid sequence, wherein the materials, or nucleotide or amino acid sequences, are foreign to each other (i.e., not found or linked together in nature).

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Particular expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

The term “promoter,” as used herein, refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon (i.e., including the 5′ untranslated region of the mRNA, typically comprising 100-200 bp). Most often the core promoter sequences lie within 1-2 kb of the translation start site, more often within 1 kbp and often within 500 bp of the translation start site. By convention, the promoter sequence is usually provided as the sequence on the coding strand of the gene it controls. In the context of this application, a promoter is typically referred to by the name of the gene for which it naturally regulates expression. A promoter used in an expression construct of the invention is referred to by the name of the gene. Reference to a promoter by name includes a wildtype, native promoter as well as variants of the promoter that retain the ability to induce expression. Reference to a promoter by name is not restricted to a particular species, but also encompasses a promoter from a corresponding gene in other species.

A polynucleotide is “heterologous” to a host cell or a second polynucleotide sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

The term “operatively linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

In some embodiments, the genetically modified bacterial host cell is an engineered Corynebacterium glutamicum strain comprising a non-native isoprenoid or isopentenol pathway that converts renewable carbon sources into a high levels tetramethyl pyrazine. C. glutamicum is one of the few microbial platforms that has been successfully scaled up in industry. A C. glutamicum mutant has been found that is able to produce alkyl pyrazines (Demain et al., 1967). However, this wild type production is a mixture and small amounts are produced over a long cultivation period.

Tetramethyl pyrazine is FDA approved for use as a coffee flavoring in the food and flavor industry for a variety of brewed and baked food products. Tetramethyl pyrazine is also known to have cognition enhancing and anti-inflammatory properties. This compound also shares chemical features with compounds that have flame retardant and resin formation properties and may have applications in the field of polymer chemistry.

In some embodiments, the genetically modified bacterial host cell is a C. glutamicum strain comprising a heterologous isoprenoid or isopentenol pathway that results in a high production of tetramethyl pyrazine. In some embodiments, the genetically modified bacterial host cell is a C. glutamicum strain comprising a heterologous isoprenoid or isopentenol pathway that results in a high production of tetramethyl pyrazine from renewable carbon sources in a period equal to or more than about 48 hours. In some embodiments, the tetramethyl pyrazine is equal to or more than about 100 mg/L of culture medium.

Tetramethyl pyrazine has known use as an FDA approved food and flavoring ingredient. It also has known uses as an antiinflammatory agent and cognitive enhancer. Finally it is the opinion of this inventor that this compound could also have properties in flame retardation, coatings and resin formation that could provide application in the polymer industry.

Tetramethyl pyrazine can be prepared from plant extracts or via chemical synthesis. While C. glutamicum has been reported to produce alkyl pyrazines in mixture at low levels over long cultivation period (Demain et al., 1967). The high production titers and rates of enriched tetramethyl pyrazine in C. glutamicum provides the foundation for a biotech application of the present invention.

In some embodiments, each enzyme is heterologous to the host cell. In some embodiments, the enzyme comprises an amino acid sequence that is at least 70%, 80%, 90%, 95%, or 99% identical to the corresponding S. cerevisae enzyme or the amino acid sequence of SEQ ID NO:1-4.

The amino acid sequence of Staphylococcus aureus MK (encoded by the mvaKl gene) is as follows:

```
(SEQ ID NO: 1)
                10 20 30 40
MTRKGYGEST GKIILIGEHA VTFGEPAIAV PFNAGKIKVL
              50 60 70 80
IEALESGNYS SIKSDVYDGM LYDAPDHLKS LVNRFVELNN
                90 100 110 120
ITEPLAVTIQ TNLPPSRGLG SSAAVAVAFV RASYDFLGKS
              130 140 150 160
LTKEELIEKA NWAEQIAHGK PSGIDTQTIV SGKPVWFQKG
              170 180 190 200
HAETLKTLSL DGYMVVIDTG VKGSTRQAVE DVHKLCEDPQ
              210 220 230 240
YMSHVKHIGK LVLRASDVIE HHNFEALADI FNECHADLKA
              250 260 270 280
LTVSHDKIEQ LMKIGKENGA LAGKLIGAGR GGSMLLLAKD
                290 300
LPTAKNIVKA VEKAGAAHTW IENLGG
```

The amino acid sequence of Corynebacterium kroppenstedtii MK (encoded by the mvaKl gene) is as follows:

```
(SEQ ID NO: 2)
                10 20 30 40
MQPLRHSDAD VITSSRRSEA PDYARLNEGQ RYKQPAEGPH
                50 60 70 80
YSALADEARG FGRAHAKIIL FGEHAVVFGE PAIAFPMQCL
                90 100 110 120
TLRATAEPCD GELWLTANNY DGPLADAPTF LSPVGATIKA
              130 140 150 160
CLDLLEYPQS GMHISCRGNV PPARGLGSSA AASAAMVDSI
              170 180 190 200
IDFSGKDVDY HSRYELVQIG ERVAHGSASG LDAHTVLNTH
              210 220 230 240
PVLFQGGRSE PITVSLGSPL VVADTGQPGD TLSAVRGVDE
              250 260 270 280
LRRTHKKRFT RNVDAIRHHT VEARIDLALD DRASLGERMN
              290 300 310 320
AVHEHLADLG VSSPELENLI SAARNAGALG AKLTGGGRGG
              330 340 350
CIIALSKDEN HAIALSDALR AAGARRTWLM HPSEFQR
```

In some embodiments, the MK, or homolog thereof, comprises an amino acid having at least 70%, 80%, 90%, 95%, or 99% amino acid identity with SEQ ID NO:1 or SEQ ID NO:2. In some embodiments, the MK, or homolog thereof, comprises an amino acid sequence within a conserved domain, such as the amino acid sequences RGLGSSAA (SEQ ID NO:5) in the GHMP kinase N domain and/or KLTGXGRGG (SEQ ID NO:6) in the GHMP kinase C domain, wherein X is any naturally occurring amino acid residue.

The amino acid sequence of Staphylococcus aureus HMGR homolog (encoded by the mvaS gene) is as follows:

```
(SEQ ID NO: 3)
                10 20 30 40
MTIGIDKINF YVPKYYVDMA KLAEARQVDP NKFLIGIGQT
                50 60 70 80
EMAVSPVNQD IVSMGANAAK DIITDEDKKK IGMVIVATES
                90 100 110 120
AVDAAKAAAV QIHNLLGIQP FARCFEMKEA CYAATPAIQL
              130 140 150 160
AKDYLATRPN EKVLVIATDT ARYGLNSGGE PTQGAGAVAM
              170 180 190 200
VIAHNPSILA LNEDAVAYTE DVYDFWRPTG HKYPLVDGAL
              210 220 230 240
SKDAYIRSFQ QSWNEYAKRQ GKSDADFASL CFHVPFTKMG
              250 260 270 280
KKALESIIDN ADETTQERLR SGYEDAVDYN RYVGNIYTGS
              290 300 310 320
LYLSLISLLE NRDLQAGETI GLFSYGSGSV GEFYSATLVE
              330 340 350 360
GYKDHLDQAA HKALLNNRTE VSVDAYETFF KRFDDVEFDE
              370 380
EQDAVHEDRH IFYLSNIENN VREYHRPE
```

The amino acid sequence of Corynebacterium kroppenstedtii HMGR homolog (encoded by the mvaA gene) is as follows:

```
(SEQ ID NO: 4)
                10 20 30 40
MSDNLYAPIP MSWIGPVHIS GNVVSGETAG WNAEDGTQNQ
                50 60 70 80
ESGASYEAVS IPMATYETPL WPSVGRGAKV SRYVEGGIRA
                90 100 110 120
TLVDERMTRS VYFEAPNAGV ALRVATELDR RRDELQAVVA
              130 140 150 160
HASRFAKLID LHVQYAGNLL FVRFEFTTGD ASGHNMVTLA
              170 180 190 200
SDNLMPWILQ QYPELRYGSI SGNYCSDKKA TAVNGILGRG
              210 220 230 240
KNVVTEMLIP RNVVEERLKT TPEQIADLNV RKNLVGTTLA
              250 260 270 280
GGLRTANAHY ANMLLGFYLA TGQDAANIVE GSQGITHAEV
              290 300 310 320
RDGDLYFSCN LPNLIVGTVG NGKGQGLEVV EENLRRLGCR
              330 340 350 360
EDRPAGDNAR RLAVLCAASV FCGELSLLAA QTNPGELMAA
HVKIERKGE
```

In some embodiments, the HMGR, or homolog thereof, comprises an amino acid having at least 70%, 80%, 90%, 95%, or 99% amino acid identity with SEQ ID NO:3. In some embodiments, the HMGR, or homolog thereof, comprises an amino acid having at least 70%, 80%, 90%, 95%, or 99% amino acid identity with SEQ ID NO:4, and optionally a conserved domain, such as the amino acid sequence TAVNGILGRGK (SEQ ID NO:7).

In some embodiments, the host cell comprises a nucleic acid encoding the one or more enzymes operatively linked to a promoter capable of expressing the one or more enzymes in the host cell. In some embodiments, the encoding of the one or more enzymes to the nucleic acid is codon optimized to the bacterial host cell. In some embodiments, the nucleic acid is vector or replicon that can stably reside in the host cell. In some embodiments, the nucleic acid is stably integrated into one or more chromosomes of the host cell.

In some embodiments, the providing step (a) comprises introducing a nucleic acid encoding the one or more enzymes operatively linked to a promoter capable of expressing the one or more enzymes in the host cell into the host cell.

In some embodiments, the culturing or growing step (b) comprises the host cell growing by respiratory cell growth. In some embodiments, the culturing or growing step (b) takes place in a batch process or a fed-batch process, such as a high-gravity fed-batch process. In some embodiments, the culture or medium comprises hydrolysates derived or obtained from a biomass, such as a lignocellulosic biomass. In some embodiments, the culture or medium comprises one or more carbon sources, such as a sugar, such as glucose or galactose, or glycerol, or a mixture thereof. In some embodiments, the carbon source is fermentable. In some embodiments, the carbon source is non-fermentable.

The present invention provides for a method for constructing a genetically modified bacterial host cell of the present invention, comprising (a) introducing a nucleic acid encoding the one or more enzymes operatively linked to a promoter capable of expressing the one or more enzymes in the host cell into the host cell.

One can modify the expression of a gene encoding any of the enzymes taught herein by a variety of methods in accordance with the methods of the invention. Those skilled in the art would recognize that increasing gene copy number, ribosome binding site strength, promoter strength, and various transcriptional regulators can be employed to alter an enzyme expression level.

### REFERENCES CITED HEREIN

Baral, N. R., Kavvada, O., Mendez-Perez, D., Mukhopadhyay, A., Lee, T. S., Simmons, B. A., Scown, C. D., 2019a. Techno-economic analysis and life-cycle greenhouse gas mitigation cost of five routes to bio-jet fuel blendstocks. Energy & Environmental Science. 12, 807-824.
Baral, N. R., Sundstrom, E. R., Das, L., Gladden, J., Eudes, A., Mortimer, J. C., Singer, S. W., Mukhopadhyay, A., Scown, C. D., 2019b. Approaches for More Efficient Biological Conversion of Lignocellulosic Feedstocks to Biofuels and Bioproducts. ACS Sustainable Chemistry & Engineering.
Chen, H., Cao, J., Zhu, Z., Zhang, G., Shan, L., Yu, P., Wang, Y., Sun, Y., Zhang, Z., 2018. A Novel Tetramethylpyrazine Derivative Protects Against Glutamate-Induced Cytotoxicity Through PGC1a/Nrf2 and PI3K/Akt Signaling Pathways. Frontiers in Neuroscience. 12.
Demain, A., Jackson, M., Trenner, N., 1967. Thiamine-dependent accumulation of tetramethylpyrazine accompanying a mutation in the isoleucine-valine pathway. Journal of bacteriology. 94, 323-326.
Dickschat Jeroen, S., Wickel, S., Bolten Christoph, J., Nawrath, T., Schulz, S., Wittmann, C., 2010. Pyrazine Biosynthesis in Corynebacterium glutamicum. European Journal of Organic Chemistry. 2010, 2687-2695.
Eng, T., Demling, P., Herbert, R. A., Chen, Y., Benites, V., Martin, J., Lipzen, A., Baidoo, E. E., Blank, L. M., Petzold, C. J., 2018. Restoration of biofuel production levels and increased tolerance under ionic liquid stress is enabled by a mutation in the essential Escherichia coli gene cydC. Microbial cell factories. 17, 159.
Ferreira, S. B., Kaiser, C. R., 2012. Pyrazine derivatives: a patent review (2008-present). Expert opinion on therapeutic patents. 22, 1033-1051.
Galperin, M. Y., Makarova, K. S., Wolf, Y. I., Koonin, E. V., 2014. Expanded microbial genome coverage and improved protein family annotation in the COG database. Nucleic acids research. 43, D261-D269.
George, A., Brandt, A., Tran, K., Zahari, S. M. N. S., Klein-Marcuschamer, D., Sun, N., Sathitsuksanoh, N., Shi, J., Stavila, V., Parthasarathi, R., 2015. Design of low-cost ionic liquids for lignocellulosic biomass pretreatment. Green Chemistry. 17, 1728-1734.
Gibson, D. G., Young, L., Chuang, R. Y., Venter, J. C., Hutchison, C. A., 3rd, Smith, H. O., 2009. Enzymatic assembly of DNA molecules up to several hundred kilobases. Nat Methods. 6, 343-5.
Goh, E. B., Baidoo, E. E., Keasling, J. D., Beller, H. R., 2012. Engineering of bacterial methyl ketone synthesis for biofuels. Appl Environ Microbiol. 78, 70-80.
Gonzalez Fernandez-Nino, S. M., Smith-Moritz, A. M., Chan, L. J., Adams, P. D., Heazlewood, J. L., Petzold, C. J., 2015. Standard flow liquid chromatography for shotgun proteomics in bioenergy research. Front Bioeng Biotechnol. 3, 44.
Hu, S., Hu, H., Mak, S., Cui, G., Lee, M., Shan, L., Wang, Y., Lin, H., Zhang, Z., Han, Y., 2018. A Novel Tetramethylpyrazine Derivative Prophylactically Protects against Glutamate-Induced Excitotoxicity in Primary Neurons through the Blockage of N-Methyl-D-aspartate Receptor. Frontiers in Pharmacology. 9.
Huerta-Cepas, J., Forslund, K., Coelho, L. P., Szklarczyk, D., Jensen, L. J., von Mering, C., Bork, P., 2017. Fast Genome-Wide Functional Annotation through Orthology Assignment by eggNOG-Mapper. Molecular Biology and Evolution. 34, 2115-2122.
Kang, A., George, K. W., Wang, G., Baidoo, E., Keasling, J. D., Lee, T. S., 2016. Isopentenyl diphosphate (IPP)-bypass mevalonate pathways for isopentenol production. Metab Eng. 34, 25-35.
Kao, T.-K., Chang, C.-Y., Ou, Y.-C., Chen, W.-Y., Kuan, Y.-H., Pan, H.-C., Liao, S.-L., Li, G.-Z., Chen, C.-J., 2013. Tetramethylpyrazine reduces cellular inflammatory response following permanent focal cerebral ischemia in rats. Experimental neurology. 247, 188-201.
Karp, P. D., Latendresse, M., Caspi, R., 2011. The pathway tools pathway prediction algorithm. Stand Genomic Sci. 5, 424-9.
Ke-ji, C., Zhen-huai, Q., Wei-Liang, W., Mu-Ying, Q., 1983. Tetramethylpyrazine in the treatment of cardiovascular and cerebrovascular diseases. Planta medica. 47, 89-89.
Keilhauer, C., Eggeling, L., Sahm, H., 1993. Isoleucine synthesis in Corynebacterium glutamicum: molecular analysis of the ilvB-ilvN-ilvC operon. Journal of bacteriology. 175, 5595-5603.
Kosuge, T., Kamiya, H., 1962. Discovery of a pyrazine in a natural product: tetramethylpyrazine from cultures of a strain of Bacillus subtilis. Nature. 193, 776.
Lee, J. L., Halogen-free flame retardant material. Apple Inc. Cupertino, Calif., US., United States, 2014.
Li, C., Knierim, B., Manisseri, C., Arora, R., Scheller, H. V., Auer, M., Vogel, K. P., Simmons, B. A., Singh, S., 2010. Comparison of dilute acid and ionic liquid pretreatment of switchgrass: Biomass recalcitrance, delignification and enzymatic saccharification. Bioresour Technol. 101, 4900-6.
Li, H.-X., Ding, M.-Y., Lv, K., Yu, J.-Y., 2001. Separation and determination of ephedrine alkaloids and tetramethylpyrazine in Ephedra sinica Stapf by gas chromatography-mass spectrometry. Journal of chromatographic science. 39, 370-374.
Liang, Q., Qi, Q., 2014. From a co-production design to an integrated single-cell biorefinery. Biotechnology advances. 32, 1328-1335.
Mao, Y., Fu, J., Tao, R., Huang, C., Wang, Z., Tang, Y.-J., Chen, T., Zhao, X., 2017. Systematic metabolic engineering of Corynebacterium glutamicum for the industrial-level production of optically pure d-(−)-acetoin. Green Chemistry. 19, 5691-5702.
Masuda, H., Mihara, S., 1988. Olfactive properties of alkylpyrazines and 3-substituted 2-alkylpyrazines. Journal of agricultural and food chemistry. 36, 584-587.
Mehmood, N., Husson, E., Jacquard, C., Wewetzer, S., Buchs, J., Sarazin, C., Gosselin, I., 2015. Impact of two ionic liquids, 1-ethyl-3-methylimidazolium acetate and 1-ethyl-3-methylimidazolium methylphosphonate, on Saccharomyces cerevisiae: metabolic, physiologic, and morphological investigations. Biotechnol Biofuels. 8, 17.
Ng, H., Seto, Keitaro, Zhang, Wei, Flame retardant vinyl addition polycycloolefinic polymers. PROMERUS, LLC (Brecksville, Ohio, US), United States, 2017.
Ouellet, M., Datta, S., Dibble, D. C., Tamrakar, P. R., Benke, P. I., Li, C., Singh, S., Sale, K. L., Adams, P. D., Keasling, J. D., Simmons, B. A., Holmes, B. M., Mukhopadhyay, A., 2011. Impact of ionic liquid pretreated plant biomass on Saccharomyces cerevisiae growth and biofuel production. Green Chemistry. 13, 2743-2749.
Peralta-Yahya, P. P., Ouellet, M., Chan, R., Mukhopadhyay, A., Keasling, J. D., Lee, T. S., 2011. Identification and microbial production of a terpene-based advanced biofuel. Nat Commun. 2, 483.
Rizzi, G. P., 1988. Formation of pyrazines from acyloin precursors under mild conditions. Journal of agricultural and food chemistry. 36, 349-352.
Ruan, Y., Zhu, L., Li, Q., 2015. Improving the electro-transformation efficiency of Corynebacterium glutamicum by weakening its cell wall and increasing the cytoplasmic membrane fluidity. Biotechnology letters. 37, 2445-2452.
Sasaki, Y., Eng, T., Herbert, R. A., Trinh, J., Chen, Y., Rodriguez, A., Gladden, J., Simmons, B. A., Petzold, C. J., Mukhopadhyay, A., 2019. Engineering Corynebacterium glutamicum to produce the biogasoline isopentenol from plant biomass hydrolysates. Biotechnology for Biofuels. 12, 41.
Stankevičiūtė, J., Vaitekūnas, J., Petkevičius, V., Gasparavičiūtė, R., Tauraitė, D., Meškys, R., 2016. Oxyfunctionalization of pyridine derivatives using whole cells of Burkholderia sp. MAK1. Scientific Reports. 6, 39129.
Wang, S., Cheng, G., Dong, J., Tian, T., Lee, T. S., Mukhopadhyay, A., Simmons, B. A., Yuan, Q., Singer, S. W., 2018. Tolerance Characterization and Isoprenol Production of Adapted Escherichia coli in the Presence of Ionic Liquids. ACS Sustainable Chemistry & Engineering. 7, 1457-1463.
Wehrs, M., Tanjore, D., Eng, T., Lievense, J., Pray, T. R., Mukhopadhyay, A., 2019. Engineering Robust Production Microbes for Large-Scale Cultivation. Trends in microbiology.
Wellerdiek, M., Winterhoff, D., Reule, W., Brandner, J., Oldiges, M., 2009. Metabolic quenching of Corynebacterium glutamicum: efficiency of methods and impact of cold shock. Bioprocess and biosystems engineering. 32, 581-592.
Xiao, Z., Hou, X., Lyu, X., Xi, L., Zhao, J.-y., 2014. Accelerated green process of tetramethylpyrazine production from glucose and diammonium phosphate. Biotechnology for biofuels. 7, 106.
Xiao, Z., Xie, N., Liu, P., Hua, D., Xu, P., 2006. Tetramethylpyrazine production from glucose by a newly isolated Bacillus mutant. Applied microbiology and biotechnology. 73, 512-518.
Yin, D., Yang, M., Wang, Y., Yin, D., Liu, H., Zhou, M., Li, W., Chen, R., Jiang, S., Ou, M., 2018. High tetramethylpyrazine production by the endophytic bacterial Bacillus subtilis isolated from the traditional medicinal plant Ligusticum chuanxiong Hort. AMB Express. 8, 193.
Zhang, Q., Zheng, X., Wang, Y., Yu, J., Zhang, Z., Dele-Osibanjo, T., Zheng, P., Sun, J., Jia, S., Ma, Y., 2018. Comprehensive optimization of the metabolomic methodology for metabolite profiling of Corynebacterium glutamicum. Applied Microbiology and Biotechnology. 102, 7113-7121.
Zhang, Z., Zhang, G., Sun, Y., Szeto, S. S. W., Law, H. C. H., Quan, Q., Li, G., Yu, P., Sho, E., Siu, M. K. W., Lee, S. M. Y., Chu, I. K., Wang, Y., 2016. Tetramethylpyrazine nitrone, a multifunctional neuroprotective agent for ischemic stroke therapy. Scientific Reports. 6, 37148.
Zhu, B.-F., Xu, Y., Fan, W.-L., 2010. High-yield fermentative preparation of tetramethylpyrazine by Bacillus sp. using an endogenous precursor approach. Journal of industrial microbiology & biotechnology. 37, 179-186.
Deng et al., 2006. Method for preparing tetramethyl pyrazine. Chinese Patent No. ZL 200310102368.0.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Production of Tetra-Methylpyrazine Using Engineered Corynebacterium glutamicum

Corynebacterium glutamicum ATCC 13032 is an established and industrially-relevant microbial host that has been utilized for the expression of many desirable bioproducts. Tetra-methylpyrazine (TMP) is a naturally occurring alkylpyrazine with broad applications spanning fragrances to resins. An engineered strain of C. glutamicum which produces 5 g/L tetra-methylpyrazine is identified and separately, a strain which can coproduce both tetra-methylpyrazine and the biofuel compound isopentenol. Ionic liquids also stimulate TMP production in engineered strains. Using a fed batch-mode feeding strategy, ionic liquid stimulated strains produced 2.2 g/L of tetra-methylpyrazine. Feedback from a specific heterologous gene pathway on host physiology leads to acetoin accumulation and the production of TMP.

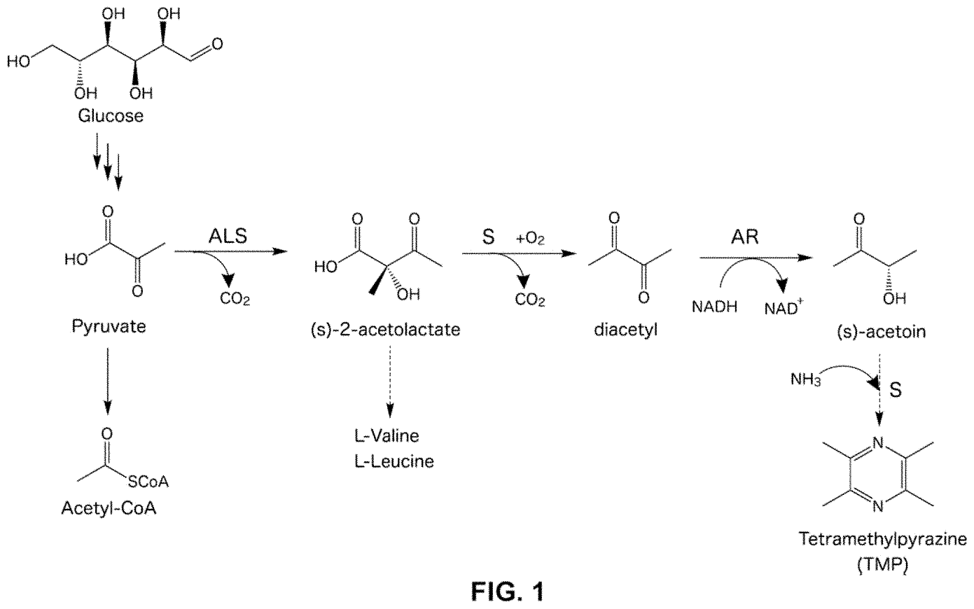

It is postulated that TMP could be produced from pyruvate via a four-step reaction (FIG. 1). The two units of the proposed immediate precursor, acetoin (Rizzi, 1988), would spontaneously react with each other to form TMP under high nitrogen concentrations (FIG. 1).

Engineered strains of C. glutamicum are examined for the production of TMP. Specific gene substitutions in a heterologous gene pathway result in strains which produce high (>5 g/L) titers of TMP or the co-production of TMP with an acetyl-CoA derived compound, isopentenol (Sasaki et al., 2019). Other factors that enhance the production of TMP in these engineered C. glutamicum strains are also described.

### Materials and Methods

### Chemicals and Reagents

All chemicals and reagents are purchased from Sigma-Aldrich (St. Louis, Mo.) or as otherwise indicated, and are of molecular biology grade or higher. When cells are cultivated in a microtiter dish format, plates are sealed with a gas-permeable film (Sigma-Aldrich, St. Louis, Mo.).

### Strain and Plasmid Construction

All strains and plasmids used are listed in Table 1 and their sequences are available at the webpage for public-registry.jbei.org. Oligo-nucleotide primers are synthesized by Integrated DNA Technologies, Inc. (San Diego, Calif.). Q5 High-Fidelity DNA Polymerase (New England Biolabs, Ipswich, Mass.) is used for polymerase chain reaction. Isothermal DNA assembly (Gibson et al., 2009) is utilized to assemble plasmids using 40 nucleotide overhangs (NEBuilder HiFi DNA Assembly Master Mix, New England Biolabs, Ipswich, Mass.). Plasmids are constructed using chemically competent E. coli DH10β (New England Biolabs). Where indicated, heterologous isopentenol production pathway is modified to incorporate both hmgr and mk homologs from S. aureus or C. kroppenstedtii, to replace the existing gene from S. cerevisiae. For S. aureus, the mk homolog is encoded by mvaKl (NCBI: WP 000197034.1) and similarly, the hmgr homolog is encoded by mvaS (NCBI: WP 045179588.1). For C. kroppenstedtii, the mk homolog is encoded by mvaKl (NCBI: ACR16826.1) and likewise, the hmgr homolog is encoded by mvaA (NCBI: WP 012730718.1). All sequences are confirmed by colony PCR and Sanger sequencing.

TABLE 1
Strains and plasmids used in this study.

| | Description | Selection | Reference |
|---|---|---|---|
| Strain | | | |
| JBEI-7936 | Corynebacterium glutamicum ATCC 13032/NHRI 1.1.2, biotin | Nx^{R} | Sasaki et al., 2019 |
| | auxotroph | | |
| JBEI-19571 | JBEI-7936 harboring p/JBEI-19559 | Kan^{R} | Sasaki et al., 2019 |
| JBEI-19652 | JBEI-7936 harboring p/JBEI-19628 | Kan^{R} | This study |
| JBEI-19658 | JBEI-7936 harboring p/JBEI-19634 | Kan^{R} | This study |
| JBEI-19566 | JBEI-7936 ΔpoxB Δ [symbol] dhA | Suc^{R}, Kan^{S} | Sasaki et al., 2019 |
| E. coli DH1 | F⁻ λ⁻ endA1 recA1 relA1 gyrA96 thi-1 glnV44 hsdR17(r_{E}⁻m_{K}⁻) | | Meselson and Yuan, 1968 |
| E. coli DH10β | F⁻ endA1 deoR⁺ recA1 galE15 galK16 [symbol] pG rpsL Δ(lac)X74 φ80lacZΔM | | Invitrogen |
| | 15 araD139 Δ(ara, leu) 7697 mcrA Δ( [symbol] -hsdRMS-mcrBC) Str^{R} λ⁻ | | |
| Plasmid | | | |
| JBEI-2600 | pEC-XK99E, E. coli-C. glutamicum shuttle expression vectors based on | Kan^{R} | Kirchner et al.. 2003 |
| | the medium-copy number plasmid including pGA1, Kan^{R}, ariV, P [symbol] | | |
| JBEI-19559 | pEC-XK99E-AK-IP-bypass | Kan^{R} | Sasaki et al., 2019 |
| JBEI-19628 | pTE221 pEC-XK99E-AK-IP-bypass-S. aureus mvaKl, mvaS | Kan^{R} | This study |
| | (substitution) | | |
| JBEI-19634 | pTE222 pEC-XK99E-AK-IP-bypass-C. kroppenstedtii mvaKl, mvaA | Kan^{R} | This study |
| | (substitution) | | |

[symbol]

indicates data missing or illegible when filed

### Growth Media Composition

Production is analyzed in several different common growth medias. Lysogeny-Broth (LB): 10 g/L tryptone, 5 g/L yeast extract, and 5 g/L NaCl. Tryptone and yeast extract are purchased from BD Biosciences (Franklin Lakes, N.J.). NCM media (Ruan et al., 2015): 17.4 g/L K₂HPO₄, 11.6 g/L NaCl, 5 g/L D-glucose, 5 g/L tryptone, 1 g/L yeast extract, 0.3 g/L trisodium citrate, 0.05 g/L MgSO₄.7H₂O, and 91.1 g/L sorbitol, pH 7.2. CGXII minimal medium is prepared as previously described (Keilhauer et al., 1993; Sasaki et al., 2019). D-glucose is used as a carbon source at the 4% (w/v) concentration or as otherwise indicated.

Preparation of Electrocompetent C. glutamicum Cells

C. glutamicum is made electrocompetent as previously described (Sasaki et al., 2019). In brief, cells are grown in NCM medium supplemented with 3% (v/v) glycine and electroporated with a Micro Pulser Electroporator (Bio-Rad Laboratories, Inc., Hercules, Calif.) at 10 μF, 600Ω, and 1800 V. After electroporation cells are immediately mixed with 400 μL of BHIS broth and heat-shocked for 6 minutes at 46° C. After a two-hour outgrowth at 30° C., cells are plated on the appropriate selective media.

Cultivation of C. glutamicum for Isopentenol and TMP Production

All cells taken from −80° C. glycerol stocks are plated on LB agar plates containing the appropriate antibiotic following standard laboratory procedures. Single colonies are inoculated and grown overnight in 5 mL LB (with antibiotics as necessary) at 30° C. on a rotary shaker at 200 rpm. Kanamycin is added to the growth media at a final concentration of 50 μg/mL. Unless otherwise noted, all seed cultures are first inoculated for growth in culture tubes. If cells are grown in a 24-well deep well format, 2 mL of culture media is used per well. Deep well plates are incubated Infors Multitron Incubator with a 3 mm Orbital Shaking Platform shaken at 999 rpm (Bottmingen, Switzerland).

To measure production of TMP or isopentenol, the adapted cultures of C. glutamicum are first back-diluted to OD₆₀₀ of 0.1 into CGXII minimal medium including 4% (w/v) D-glucose at the concentrations described above. Cells from a seed culture are sub-cultured twice to adapt cells to growth in the media as previously described (Sasaki et al., 2019). The production pathway is then induced as before when cultures reached an OD₆₀₀ of ˜0.8. Exogenous ionic liquids are added to the adapted cultures at the same time of induction with IPTG.

### Analytical Methods for Chemical Identification and Quantification

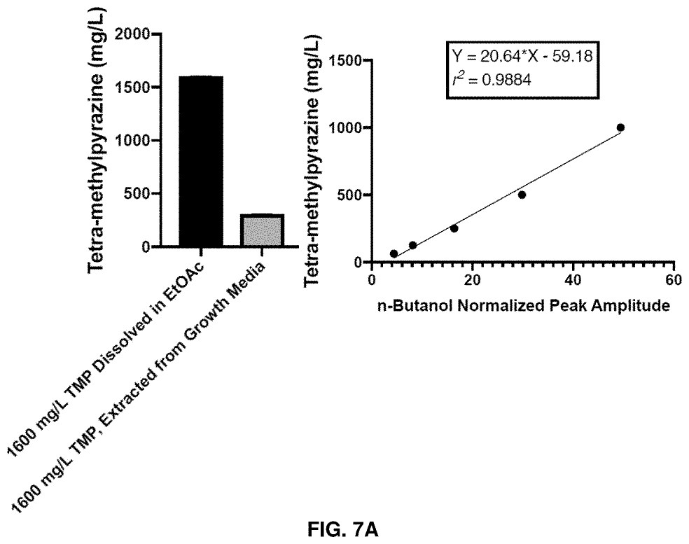

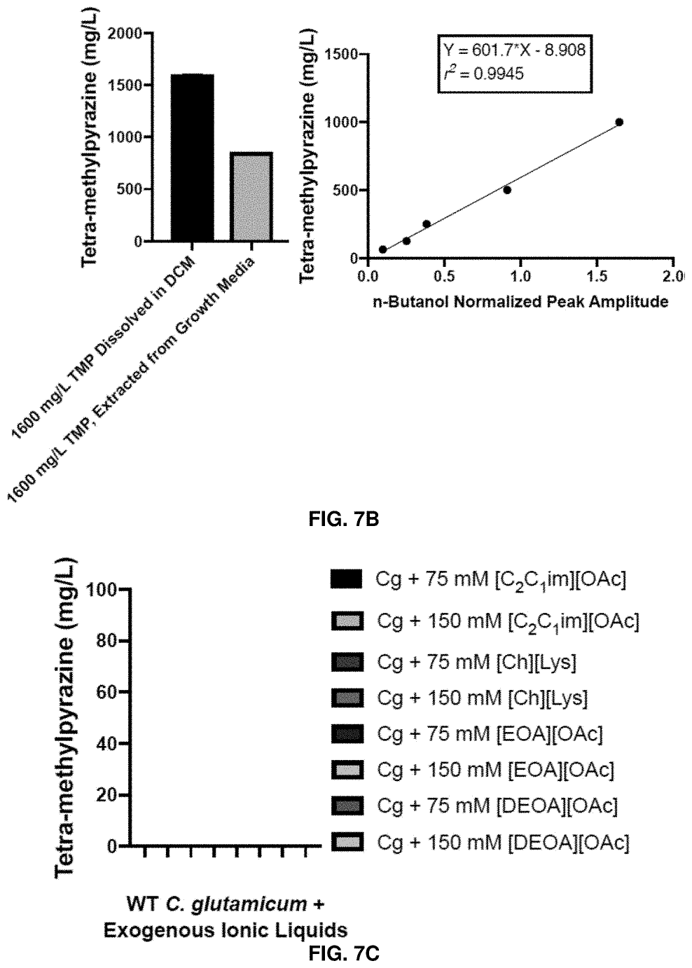

For metabolite quantification, 300 μL of cell culture media is combined with 300 μL of ethyl acetate containing n-butanol (10 μg/L) as an internal standard and processed as described previously (Goh et al., 2012; Kang et al., 2016; Sasaki et al., 2019). Briefly, samples are shaken at maximum speed for 15 minutes using an MT-400 microtube mixer (TOMY Seiko, Tokyo, Japan) and then centrifuged at 14,000 g for 3 mins to separate the organic phase from the aqueous phase. 60 μL of the organic layer is transferred into a GC vial and 1 μL is analyzed by Agilent GCMS equipped with a DB-5 column (Agilent Technologies, Santa Clara, Calif., USA) or Thermo GC-FID equipped with a DB-WAX column (Agilent Technologies, Santa Clara, Calif., USA) for quantification of TMP, acetoin, diacetyl, and isopentenol (3-methyl-3-buten-1-ol). Analytical grade standards are purchased from Sigma-Aldrich (St. Louis, Mo.) and used to calculate analyte concentrations and confirm identification of peaks. To compare the extraction efficiency of TMP into dichloromethane, the same protocol as above is used where dichloromethane is used in place of ethylacetate as the extraction solvent. Reported TMP titers are calculated using a linear curve of TMP peak areas normalized to n-butanol generated from authentic standards resuspended directly into ethyl acetate. Values are corrected for inefficient extraction from CGXII media into ethyl acetate by multiplying GCFID values by 5.88 (refer to FIG. 7A).

To determine the spontaneous conversion rate of acetoin or diacetyl to tetra-methylpyrazine, pure 100 mM of pure analytical grade standards are added to CGXII media supplemented with 4% D-glucose. These cultures are then incubated at 30° C. as described in the section on “Cultivation of C. glutamicum for Isopentenol and TMP production” for 48 hours, after which samples are harvested for ethyl acetate extraction and quantification by GC-FID. Conversion of acetoin or diacetyl to TMP is quantified using authentic standards, and samples are tested in triplicate.

A commonly used extraction solvent, ethyl acetate, shows a 17% extraction efficiency for TMP from CGXII culture media (FIG. 7A). While toxic and more challenging to handle, dichloromethane shows a higher extraction efficiency for TMP at 50% from culture media (FIG. 7B).

Comparison of Pathway Protein Abundance in E. coli and C. glutamicum

E. coli (DH1) and C. glutamicum (ATCC 13032) strains harboring plasmid pTE220 are cultivated as described in the section on “Cultivation of C. glutamicum for Isopentenol and TMP production”, but grown in LB media supplemented with 56 mM glucose and 50 μg/mL Kanamycin+500 μM IPTG to ensure an equitable comparison between these two hosts. Crude cell extracts containing are prepared exactly as described in (Eng et al., 2018) and analyzed with an Agilent 6550 iFunnel Q-TOF mass spectrometer (Agilent Technologies, Santa Clara, Calif.) coupled to an Agilent 1290 UHPLC system, with a method as described previously (Gonzalez Fernandez-Nino et al., 2015). To measure global proteomic changes between pathway variants in C. glutamicum, we analyze the set of proteins absent in wild-type C. glutamicum but detected in the pathway variant samples by shotgun proteomics, filtering for proteins with at least two unique total peptides detected. Endogenous proteins are functionally annotated using eggNOG-mapper (Huerta-Cepas et al., 2017) to assign COG annotations (Galperin et al., 2014). Hierarchical clustering using a one minus pearson correlation is calculated for both proteins and strains using the Morpheus software package (webpage for: software.broadinstitute.org/morpheus).

### Fed-Batch Production of TMP in a 2 L Bioreactor Format

Fed-batch production is performed using a 2 L bioreactor equipped with a Sartorius BIOSTAT B plus control unit for regulating dissolved oxygen (DO), pH, and temperature. A seed train is used to generate the starting inoculum for the bioreactor, which is electronically controlled to a pH of 7.0+/−0.3 using 7.5 M ammonium hydroxide and 4 M sulfuric acid. The temperature of the bioreactor is kept constant at 30° C. throughout the production time course. Diluted 10% (v/v) PEG-PPG-PEG, Poly(ethylene glycol)-block-polypropylene glycol)-block-poly(ethylene glycol) (Sigma-Aldrich) is added as needed to control foaming. During the initial growth phase, DO is controlled at 30% saturation by varying agitation speed from 400 to 1,200 rpm, and then the air-flow rate is subsequently varied from 0.5-1.5 volume of air per volume of medium per minute (vvm). Cultures are induced with 500 μM IPTG after 3 hours of the batch phase. After depletion of the starting D-glucose (˜10 g/L), feeding is initiated in low oxygen conditions by dropping DO levels down to 0-5% of saturation by varying agitation speed from 400 to 750 rpm and gassing with 0.25 vvm, and the feeding rate is controlled to keep the D-glucose concentration above 10 g/L. The feed solution contains 500 g/L D-glucose, 5 g/L (NH₄)₂SO₄, 1.5 g/L KH₂PO₄, 1.5 g/L K₂HPO₄, 5 g/L yeast extract, 0.5 mM IPTG, and kanamycin. At specific time points, 5 mL samples are collected from the bioreactor by syringe affixed to the sampling tube and used for growth, GCFID, and HPLC analysis.

### Results

### Engineered Strains Harboring a Heterologous Gene Pathway Produce Tetra-Methylpyrazine

The formation of tetra-methylpyrazine (TMP) is observed while analyzing heterologous gene pathway variants expressed in engineered C. glutamicum strains. C. glutamicum was previously engineered for the production of isopentenol using a heterologous, mevalonate-based five gene pathway, referred to as the “original” pathway (Sasaki et al., 2019). Pathway variants are constructed because two of the genes (mk and hmgR derived from Saccharomyces cerevisiae) are poorly expressed under standard laboratory growth conditions. These variants are constructed by substituting mk and hmgR gene homologs from either Staphylococcus aureus or Corynebacterium kroppenstedtii into the heterologous gene pathway and assayed for TMP production over a 48 hour time course.

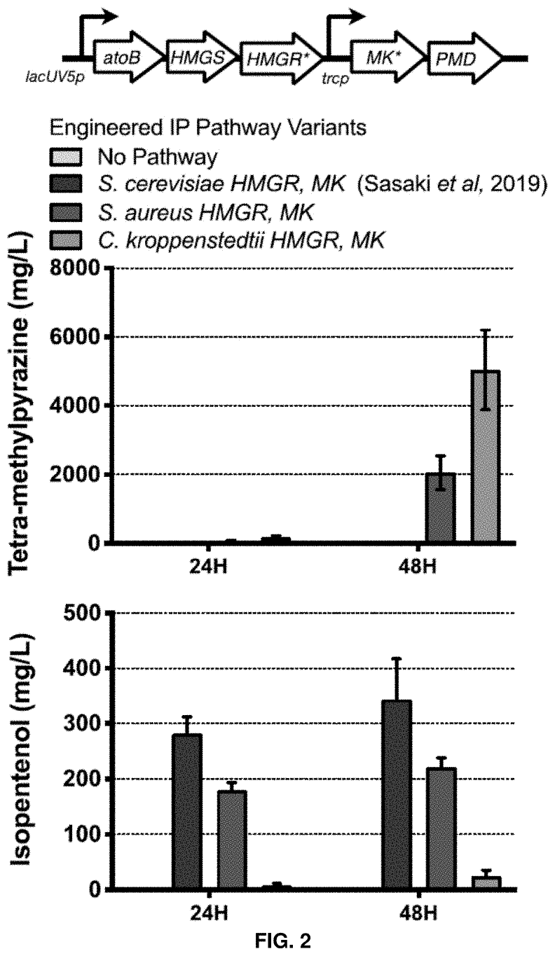

These strains with two successfully expressed mk homologs (from S. aureus and C, kroppenstedtii) and hmgR from S. aureus result in the production of TMP as detected by GC analysis (FIG. 2). In the absence of a heterologous gene pathway, no new products are detected. The C. glutamicum strain harboring the original pathway produces 300 mg/L isopentenol and did not produce detectable TMP (FIG. 2). In contrast to the original pathway, the S. aureus Mk, HmgR pathway variant produces 2.2 g/L of TMP and ˜200 mg/L of isopentenol (FIG. 2). Furthermore, the C. kroppenstedtii Mk, HmgR pathway variant produces 5 g/L of TMP and did not produce isopentenol (FIG. 2). These results indicate that the choice of pathway variant expressed in C. glutamicum impacts the amount of TMP produced.

It is examined if candidate TMP pathway proteins (FIG. 1) increases expression in these two new strains, but do not detect significant differences in protein abundance for the three candidate pathway genes, IlvB, IlvN, or ButA among the strain variants. As there is no detectable change in protein abundance for the pathway genes, proteome-wide changes to cellular metabolism which could favor spontaneous conversion to TMP is searched.

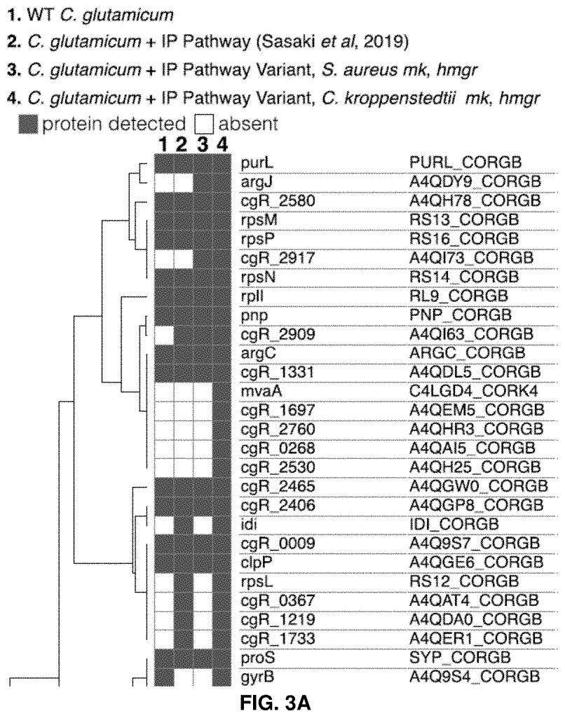

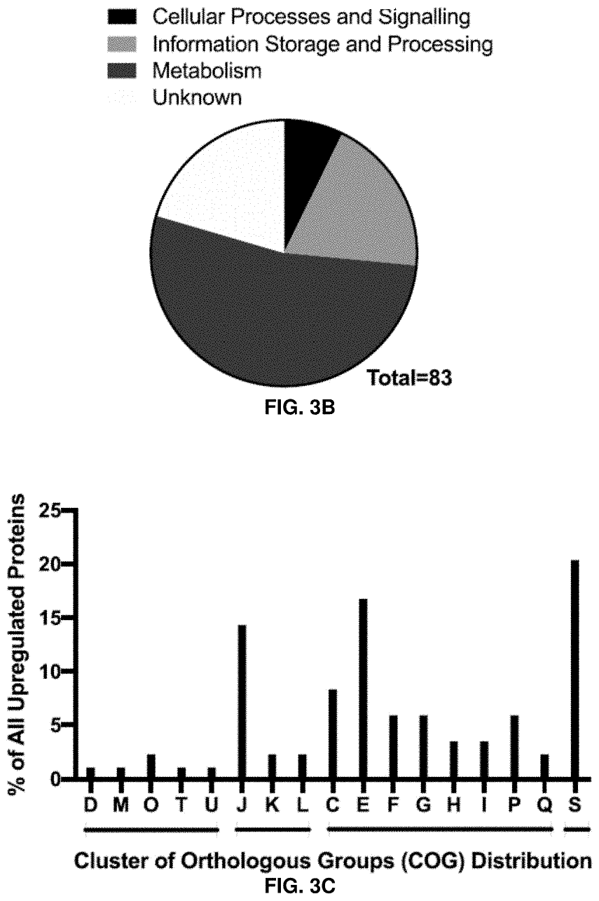

Four genes involved in the tryptophan synthesis pathways (TrpE/CgR 2916, TrpG/CgR 2917, TrpB/CgR 2920, TrpA/CgR 2921) are newly detected in strains expressing the isopentenol pathway variants from S. aureus or C. kroppenstedtii (FIGS. 3A to 3C). Additionally, two genes involved in nitrogen metabolism (ArgJ/CgR 1458, CgR 1470) are also identified as enriched in these strains. A systems level analysis (Galperin et al., 2014) of the 83 upregulated proteins indicates that 53% of these genes are involved in cellular physiology, many relate to amino acid metabolism and transport (FIGS. 3A to 3C). Complete data of all enriched proteins from these engineered strains is plotted (data not shown). As the expression of specific heterologous gene pathways favored nitrogen-accumulating processes that could enhance TMP production (Rizzi, 1988), it is reasonable that the enrichment of new compounds could be favored in these variant strain backgrounds.

### Analysis of Tetra-Methylpyrazine Biosynthesis and its Downstream Extraction

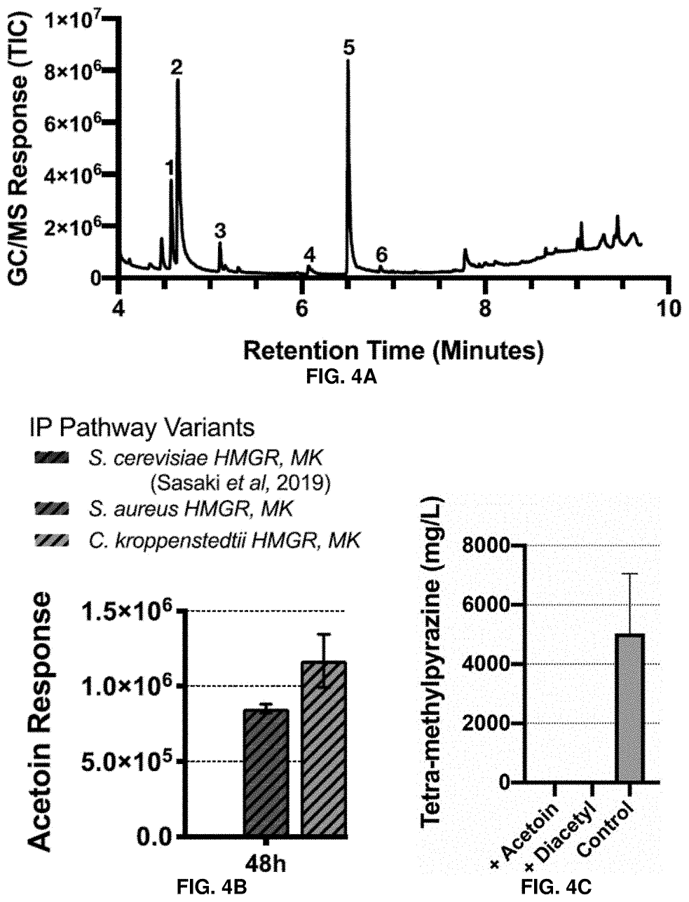

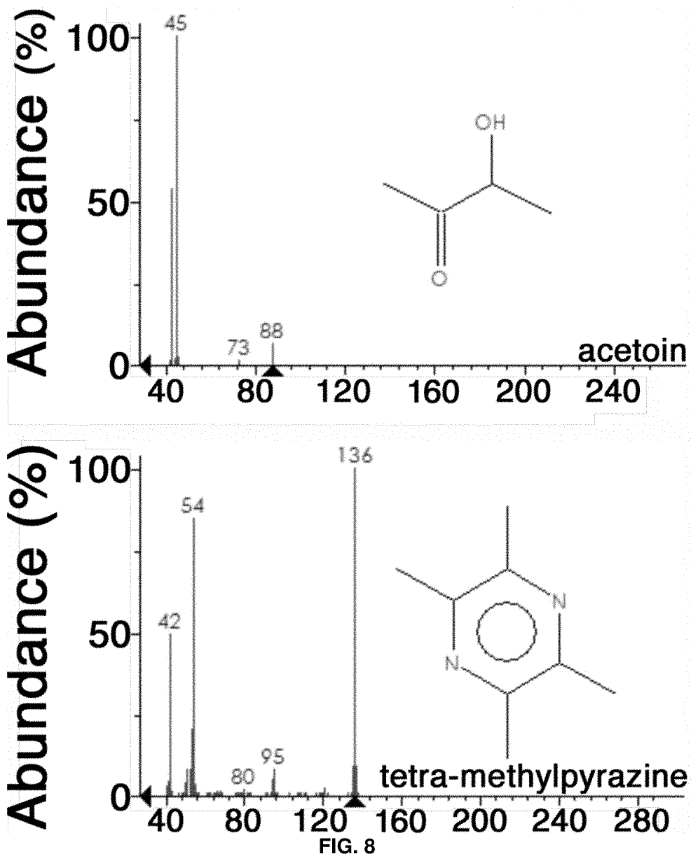

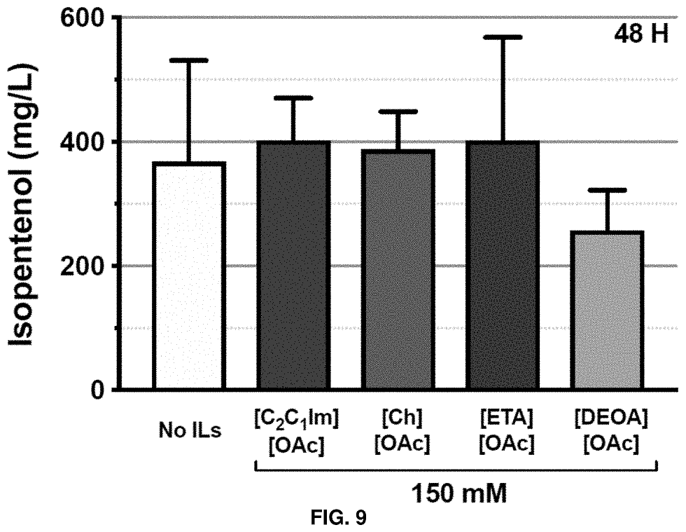

To understand how TMP might be produced in these strains, the GC/MS trace files from these samples are examined to determine if other related pathway intermediates might also be detected. GC/MS analysis of the additional analytes indicates the main additional peak to be tetra-methylpyrazine, TMP (FIG. 4A peak no. 5 and FIG. 8). The accumulation of tri-methylpyrazine and 3,5-diethyl-2-methyl-pyrazine is also detected (FIG. 2, peak no. 4 and peak no. 6).

Consistent with the proposed pathway for pyrazine synthesis (FIG. 1), S-Acetoin (3-Hydroxybutanone; FIG. 4A peak no. 1 and FIG. 8), the proposed precursor of tri/tetra-methylpyrazine, is also detected (Karp et al., 2011; Xiao et al., 2014). S-Acetoin is specifically identified in samples with detectable TMP production levels and absent in samples which does not produce TMP (FIG. 4B). The accumulation of acetoin in these strains strengthens the evidence in support of the proposed biological route to produce TMP.

It is examined if TMP can form spontaneously from acetoin or diacetyl in the absence of a microbial host under biologically relevant cultivation conditions. Commercially purchased authentic standards for acetoin and diacetyl are spiked into CGXII media and tested for formation of TMP after the same cultivation time period of 48 hours. Spontaneous conversion of either acetoin or diacetyl to TMP is not detected, suggesting that the reaction most efficiently produces TMP in the presence of a cell (FIG. 4C).

### Ionic Liquids, a Renewable Pretreatment Reagent, Enhances Production of Tetra-Methylpyrazine

Most sustainable production requires the use of renewable carbon sources such as plant derived biomass and pose trade-offs due to the incompatibilities between processes (Baral et al., 2019b; Eng et al., 2018; Ouellet et al., 2011; Sasaki et al., 2019; Wang et al., 2018). The behavior of C. glutamicum strains when cultivated with reagents used in the pretreatment of sustainable carbon streams is evaluated. Specifically, it is asked if trace levels of ionic liquids, a promising reagent for plant biomass deconstruction (George et al., 2015; Li et al., 2010), changes the amount of TMP produced in strains expressing the original IP pathway.

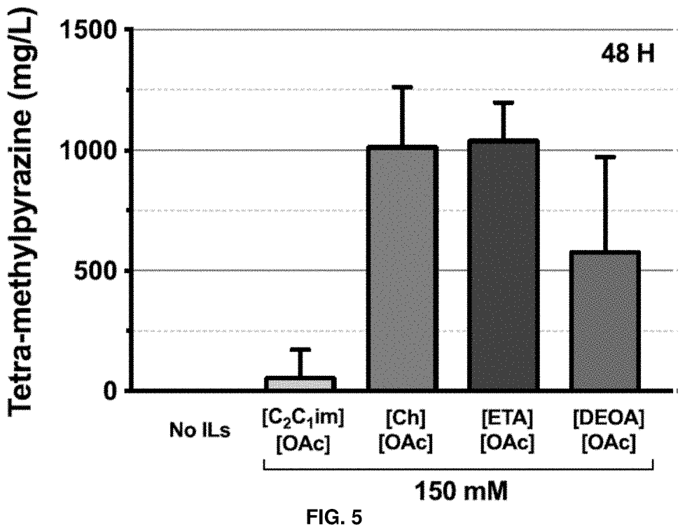

The effect of three different classes of ionic liquids is characterized on the behavior of engineered C. glutamicum strains for the production of TMP. The ionic liquids chosen are acetate salts with 1-ethyl-3-methyl imidazolium ([C₂C₁im]⁺); cholinium ([Ch]⁺); ethanolamine [ETA]; and diethanolamine [DEOA], [ETA] and [DEOA] cations. [C₂C₁im] and [Ch] are two biogenic ILs whereas [ETA] and [DEOA] are two representative protic ILs. TMP is detected at the 48 hour time point under these ILs-stressed conditions (FIG. 5). In the presence of exogenous 150 mM [C₂C₁im][OAc], 63 mg/L TMP is detected (FIG. 5, gray bar). Interestingly, in contrast to bioproduction in other microbial hosts (Eng et al., 2018; Ouellet et al., 2011) inhibition of production in the presence of ILs is not observed. Rather, when treated with 150 mM [Ch][OAc], higher levels of TMP production are detected, with titers reaching 1021 mg/L (FIG. 5, blue bar). Of the protic ILs, treatment with 150 mM [ETA][OAc] produces a similar titer to [Ch][OAc], with measured titers around 1050 mg/L (FIG. 5, green bar). 150 mM [DEOA][OAc] treatment results in an accumulation of TMP to 585 mg/L (FIG. 5, orange bar). When expressed in C. glutamicum, the original IP pathway does not produce TMP under standard cultivation conditions, but treatment with specific ILs result in TMP production from undetectable to over 1 g/L.

Ionic liquids alone are not sufficient to induce TMP production in wild-type C. glutamicum (FIG. 7C). The amount of TMP produced in response to the IL is more variable when treating cells with 75 mM ILs, but TMP still accumulates in the same set of ILs. These strains also co-produce TMP and ˜350 mg/L isopentenol using the unmodified isopentenol production pathway (FIG. 9). These results provide a new perspective on using ILs as pretreatment reagents. Rather than framing ILs as pretreatment contaminants that must be removed, trace ionic liquids remaining from biomass pretreatment regimes can be beneficial as inducers allowing the coproduction of TMP and isopentenol.

### Production of TMP in Fed Batch Mode Cultures

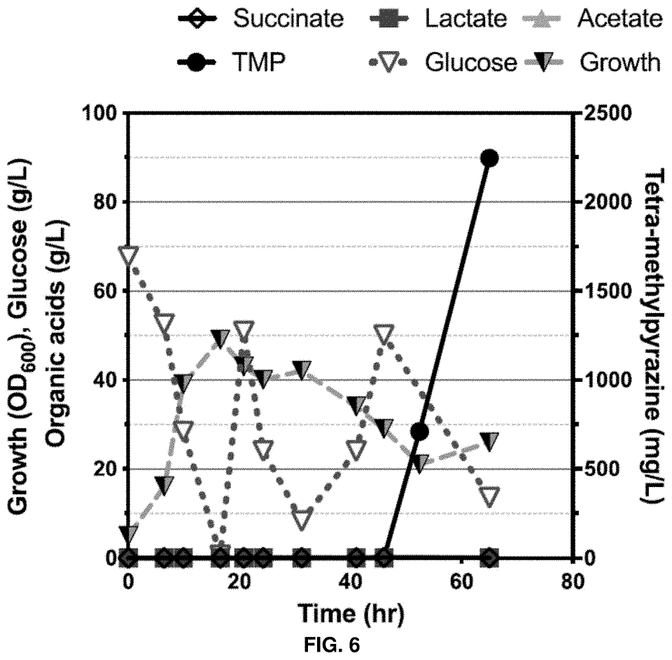

Having observed the accumulation of TMP in our engineered strains, it is next determined if TMP production can be produced under more industrially relevant cultivation conditions. The cultivation format is changed from a 24 well deep well microtiter dish to a 2 L bioreactor format in fed batch mode. To retain flexibility in the choice of final product, 50 mM of the IL [Ch][Lys] is added to stimulate TMP production in the engineered C. glutamicum strain using the original pathway. In fed batch mode, the rate of TMP production is slower what is observed in batch mode, as only detect 700 mg/L of TMP is detected after 48 hours of cultivation (FIG. 6, compare to time course in FIG. 2). It is possible that TMP is only produced after nutrient exhaustion in stationary phase, as only TMP production is detected 6 hours after the final addition of feed solution. The TMP titer after 65 hours cultivation reaches 2 g/L (FIG. 6), which is higher than the final titer in the smaller lab-scale format using IL-stimulated production (compare with FIG. 5). Isopentenol production under these cultivation conditions is near the detection limit (5 mg/L) potentially due to the increased aeration from the impeller driven mixing inherent to cell growth conditions in the bioreactor (FIG. 6). Phasing separate isopentenol with a dodecane overlay (Peralta-Yahya et al., 2011) is attempted, but dodecane inhibits growth of C. glutamicum under these conditions. These results indicate the scalability of TMP production under industrially relevant formats, but further work would be required to co-produce isopentenol.

### DISCUSSION

This report illustrates two parameters by which C. glutamicum can be modified to produce tetra-methylpyrazine. The highest final titers from batch and fed-batch production modes compare favorably with published titers from Bacillus subtilis (Xiao et al., 2014; Yin et al., 2018) and does not require a post-cultivation denaturing step to produce TMP. Unlike E. coli, C. glutamicum responds to the burden of expressing a heterologous IP pathway by accumulating TMP. A previous report had indicated that redirecting central metabolism in C. glutamicum away from acetate led to the accumulation of high intracellular levels of acetoin (Mao et al., 2017), but did not report TMP in their strains. This result from Mao et al. is consistent with the hypothesis, in which the burden of a specific heterologous gene pathway can instead favor high level TMP accumulation. It is likely that there is some interaction between the heterologous gene pathway and native metabolism at the metabolite level. Efficient and universal metabolite quenching for metabolic flux analysis could be utilized to examine this possibility (Wellerdiek et al., 2009; Zhang et al., 2018).

Certain ionic liquids are known to impact cellular morphology and physiology (Mehmood et al., 2015). Their impact on engineered strains can also be pathway-specific; in E. coli, the presence of an isopentenol production pathway (but not a limonene producing pathway) can funnel production away from the desired molecule towards acetate (Eng et al., 2018). While additional studies are required to understand the beneficial impact of ionic liquids on the production of TMP in engineered C. glutamicum strains, this study provides an alternate paradigm of ILs as a useful tool for improving production instead of merely being an unwanted contaminant. The added burden of ionic liquids could be a new metric for assessing how a heterologous gene pathway can impinge upon native metabolism, especially when evaluating hosts originating from different environmental niches and primary carbon sources (Wehrs et al., 2019).

The production of TMP is correlated with the accumulation of acetoin, providing additional evidence to strengthen its proposed biosynthetic pathway (FIG. 1). As spontaneous accumulation of TMP from either acetoin or di-acetyl when added directly to culture media is not detected, this reaction must require the cellular environment to occur. These observations highlight the importance of considering both the specific heterologous gene pathway as well as potential feedback on the chosen microbial host. With high titers of TMP, these C. glutamicum strains will facilitate rapid evaluation of TMP derivatives using candidate enzyme libraries to be expressed in a gram-positive microbial host (Hu et al., 2018; Stankevičiūtė et al., 2016; Zhang et al., 2016). Being able to produce >1 g/L quantities of many TMP derivatives will unlock the potential of this emerging molecule for a broader spectrum of applications.

Besides the exclusive production of TMP, examples of the coproduction of two different final products from the same microbial host can also be of value (Liang and Qi, 2014). As these two compounds have distinct applications, such an industrial process would likely require refinement of existing purification techniques for efficient separation. Isopentenol easily partitions into organic solvents such as ethyl acetate, and TMP can be precipitated from the aqueous phase after cooling to ˜4° C. (Xiao et al., 2006). Regardless, this report is the first instance in which either TMP or isopentenol has been the final product for a co-production study. The use of TMP as a possible coproduced compound with isopentenol will allow more nuance and control in the technoeconomic analysis of scaling up either product (Baral et al., 2019a; Baral et al., 2019b).

### CONCLUSIONS

In summary, herein describes the successful use of the gram-positive industrial microorganism, C. glutamicum, for the high titer (>5 g/L) production of TMP. These strains can also be used to coproduce TMP as well as the biofuel candidate, isopentenol. Production of TMP was scaled-up to industrially relevant conditions in a 2 L fed batch bioreactor, where we observed 2 g/L TMP when cells are treated with exogenous ionic liquid.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A genetically modified bacterial host cell capable of producing tetramethyl pyrazine comprising one or more enzymes of a heterologous isoprenoid or isopentenol production pathway.

2. The genetically modified bacterial host cell of claim 1, wherein the genetically modified bacterial host cell is also capable of producing isopentenol.

3. The genetically modified bacterial host cell of claim 1, wherein the genetically modified bacterial host cell is Corynebacterium cell.

4. The genetically modified bacterial host cell of claim 3, wherein the genetically modified bacterial host cell is Corynebacterium glutamicum.

5. The genetically modified bacterial host cell of claim 4, wherein the Corynebacterium glutamicum is strain ATCC 13032.

6. The genetically modified bacterial host cell of claim 1, wherein the one or more enzymes of a heterologous isoprenoid or isopentenol pathway comprise the enzymes: acetyl-CoA acetyltransferase (AtoB), hydroxymethylglutaryl-CoA synthase (HMGS), 3-hydroxy-3-methylglutaryl-CoA reductase (HMGR), mevalonate kinase (MK), and/or phosphomevalonate decarboxylase (PMD), phosphatase, or any homolog thereof having the same enzymatic activity.

7. The genetically modified bacterial host cell of claim 6, wherein the HMGR, or homolog thereof having the same enzymatic activity, comprises an amino acid having at least 70% amino acid identity with SEQ ID NO:3 or SEQ ID NO:4.

8. The genetically modified bacterial host cell of claim 7, wherein the HMGR, or homolog thereof having the same enzymatic activity, comprises an amino acid having at least 70% amino acid identity with SEQ ID NO:4, and a conserved domain having an amino acid sequence TAVNGILGRGK (SEQ ID NO:7).

9. The genetically modified bacterial host cell of claim 6, wherein the MK, or homolog thereof having the same enzymatic activity, comprises an amino acid having at least 70% amino acid identity with SEQ ID NO:1 or SEQ ID NO:2.

10. The genetically modified bacterial host cell of claim 9, wherein the MK, or homolog thereof having the same enzymatic activity, comprises an amino acid sequence RGLGSSAA (SEQ ID NO:5) in a GHMP kinase N domain and/or KLTGXGRGG (SEQ ID NO:6) in a GHMP kinase C domain, wherein X is any naturally occurring amino acid residue.

11. A method for a genetically modified bacterial host cell producing tetramethyl pyrazine (TMP), comprising (a) providing a genetically modified bacterial host cell of claim 1, (b) culturing or growing the host cell in a suitable culture or medium such that TMP is produced and optionally isopentenol is produced, and (c) optionally extracting or separating the TMP and optionally isopentenol from the rest of the culture or medium, and/or host cell.

12. A method for constructing a genetically modified bacterial host cell of claim 1, comprising (a) introducing a nucleic acid encoding the one or more enzymes of a heterologous isoprenoid or isopentenol production pathway operatively linked to a promoter capable of expressing the one or more enzymes of a heterologous isoprenoid or isopentenol production pathway in the host cell into the host cell.


SEQUENCE LISTING (7 sequences)

SEQ ID NO: 1 - protein, 306 aa, Staphylococcus aureus
MTRKGYGESTGKIILIGEHAVTFGEPAIAVPFNAGKIKVLIEALESGNYSSIKSDVYDGMLYDAPDHLKSLVNRFVELNNITEPLAVTIQTNLPPSRGLGSSAAVAVAFVRASYDFLGKSLTKEELIEKANWAEQIAHGKPSGIDTQTIVSGKPVWFQKGHAETLKTLSLDGYMVVIDTGVKGSTRQAVEDVHKLCEDPQYMSHVKHIGKLVLRASDVIEHHNFEALADIFNECHADLKALTVSHDKIEQLMKIGKENGAIAGKLTGAGRGGSMLLLAKDLPTAKNIVKAVEKAGAAHTWIENLGG

SEQ ID NO: 2 - protein, 357 aa, Corynebacterium kroppenstedtii
MQPLRHSDADVITSSRRSEAPDYARLNEGQRYKQPAEGPHYSALADEARGFGRAHAKIILFGEHAVVFGEPAIAFPMQCLTLRATAEPCDGELWLTANNYDGPLADAPTFLSPVGATIKACLDLLEYPQSGMHISCRGNVPPARGLGSSAAASAAMVDSIIDFSGKDVDYHSRYELVQIGERVAHGSASGLDAHTVLNTHPVLFQGGRSEPITVSLGSPLVVADTGQPGDTLSAVRGVDELRRTHKKRFTRNVDAIRHHTVEARIDLALDDRASLGERMNAVHEHLADLGVSSPELENLISAARNAGALGAKLTGGGRGGCIIALSKDENHAIALSDALRAAGARRTWLMHPSEFQR

SEQ ID NO: 3 - protein, 388 aa, Staphylococcus aureus
MTIGIDKINFYVPKYYVDMAKLAEARQVDPNKFLIGIGQTEMAVSPVNQDIVSMGANAAKDIITDEDKKKIGMVIVATESAVDAAKAAAVQIHNLLGIQPFARCFEMKEACYAATPAIQLAKDYLATRPNEKVLVIATDTARYGLNSGGEPTQGAGAVAMVIAHNPSILALNEDAVAYTEDVYDFWRPTGHKYPLVDGALSKDAYIRSFQQSWNEYAKRQGKSLADFASLCFHVPFTKMGKKALESIIDNADETTQERLRSGYEDAVDYNRYVGNIYTGSLYLSLISLLENRDLQAGETIGLFSYGSGSVGEFYSATLVEGYKDHLDQAAHKALLNNRTEVSVDAYETFFKRFDDVEFDEEQDAVHEDRHIFYLSNIENNVREYHRPE

SEQ ID NO: 4 - protein, 369 aa, Corynebacterium kroppenstedtii
MSDNLYAPIPMSWIGPVHISGNVVSGETAGWNAEDGTQNQESGASYEAVSIPMATYETPLWPSVGRGAKVSRYVEGGIRATLVDERMTRSVYFEAPNAGVALRVATELDRRRDELQAVVAHASRFAKLIDLHVQYAGNLLFVRFEFTTGDASGHNMVTLASDNLMPWILQQYPELRYGSISGNYCSDKKATAVNGILGRGKNVVTEMLIPRNVVEERLKTTPEQIADLNVRKNLVGTTLAGGLRTANAHYANMLLGFYLATGQDAANIVEGSQGITHAEVRDGDLYFSCNLPNLIVGTVGNGKGQGLEVVEENLRRLGCREDRPAGDNARRLAVLCAASVFCGELSLLAAQTNPGELMAAHVKIERKGE

SEQ ID NO: 5 - protein, 8 aa, Staphylococcus aureus
RGLGSSAA

SEQ ID NO: 6 - protein, 9 aa, Staphylococcus aureus
  misc_feature (5)..(5): Xaa can be any naturally occurring amino acid
KLTGXGRGG

SEQ ID NO: 7 - protein, 11 aa, Corynebacterium kroppenstedtii
TAVNGILGRGK